(19) United States
(12) Patent Application Publication
(10) Pub. No.: US 2013/0149331 A1
(43) Pub. Date: Jun. 13, 2013

(54) RHAMNOSE AND FORSSMAN CONJUGATED IMMUNOGENIC AGENTS

(71) Applicants: Peng George Wang, Alpharetta, GA (US); Wenlan Alex Chen, Ankeny, IA (US); Brian Martin, Ankeny, IA (US); Mario R. Mautino, Ankeny, IA (US); Nicholas N. Vahanian, Polk City, IA (US); Charles J. Link, JR., Clive, IA (US)

(72) Inventors: Peng George Wang, Alpharetta, GA (US); Wenlan Alex Chen, Ankeny, IA (US); Brian Martin, Ankeny, IA (US); Mario R. Mautino, Ankeny, IA (US); Nicholas N. Vahanian, Polk City, IA (US); Charles J. Link, JR., Clive, IA (US)

(21) Appl. No.: 13/463,420

(22) Filed: May 3, 2012

Related U.S. Application Data
(60) Provisional application No. 61/482,011, filed on May 3, 2011.

(51) Int. Cl. A61K 47/48

(52) U.S. Cl. CPC A61K 47/48092
USPC 424/193.1; 536/123.13; 536/1.11; 435/325; 530/322; 435/188; 530/395; 530/358

(57) ABSTRACT

The present invention provides an immunogenic composition comprising a T-cell antigen in association with a rhamnose monosaccharide and/or Forssman disaccharide, and corresponding methods for inducing immune response. The T-cell antigen may be for example, a tumor vaccine, such as a tumor cell or one or more tumor antigens. The invention takes advantage of the naturally high titers of anti-Rhamnose and/or anti-Forssman disaccharide in humans to target vaccine compositions to antigen presenting cells.

### CROSS-REFERENCE TO RELATED APPLICATIONS

This Application claims the benefit of U.S. Provisional Application No. 61/482,011, filed May 3, 2011, which is hereby incorporated by reference in its entirety.

### STATEMENT REGARDING FEDERALLY SPONSORED RESEARCH OR DEVELOPMENT

The invention described herein was supported by funding from the National Institutes of Health Grant Numbers R21AI083513 and R01AI083754. The Government has certain rights.

### FIELD OF THE INVENTION

The invention relates to, among other things, saccharide-conjugated immunogenic agents, and their uses for inducing immune response.

### BACKGROUND

The targeting of autologous vaccines towards antigen presenting cells (APCs) via the in vivo complexation between anti α-Gal (anti-Gal) antibodies and α-Gal antigens presents a promising cancer immunotherapy with enhanced immunogenicity. This strategy takes advantage of the ubiquitous anti-Gal antibody in human serum. Some studies have suggested that the promising properties of α-Gal are not shared by all saccharide antigens that also boast high titers in humans. Indeed, preliminary studies suggested that rhamnose-conjugation does not enhance antigen presentation. See, S Lombardo, Rhamnose as a Tumor-Antigen Immunogen, Proceedings of the Thirtieth Annual Sigma Xi Student Research Symposium, University of Toledo (Fall 2009).

### SUMMARY OF THE INVENTION

The present invention provides an immunogenic composition comprising an antigen in association with a rhamnose and/or Forssman epitope, and provides corresponding methods for inducing immune response. The invention takes advantage of the naturally high titers of anti-Rhamnose and/or anti-Forssman in humans to effectively target vaccine compositions to antigen presenting cells. In addition, the low titers of such antibodies in wildtype mice provide for a convenient tool for developing immunotherapies in accordance with the invention.

In various embodiments the compositions and methods of the invention employ attenuated vaccine tumor cells, on which rhamnose monosaccharide and/or Forssman disaccharide are present. In various embodiments, the rhamnose monosaccharide or Forssman disaccharide is conjugated to the tumor cells or viral-infected cells or virus-like particles, or isolated or synthetic tumor antigen(s) or viral antigens via a chemical linker. The artificial glycoconjugate can allow for or facilitate intracellular processing of the antigen for effective presentation on APCs through the formation of immuno-complexes with pre-existing naturally acquired anti-rhamnose or anti-Forssman antibodies, respectively.

The present invention provides compositions and methods of use to target peptide antigens, for example, those of either viral or tumor associate origin, including autologous antigens, to antigen presenting cells for effective presentation to the immune system. The invention in certain embodiments, overcomes limitations associated with the development of protein or peptide vaccines, including with autologous antigens. For example, tumor associated antigens (TAA), TAA-derived peptides, or viral antigens (VA) can be modified by functionalization with an L-Rhamnose monosaccharide or Forssman disaccharide epitope which promotes the in vivo formation of immunocomplexes with natural anti-Rham or anti-Forssman antibodies. The binding of natural IgG or IgM to the saccharide epitopes facilitates the formation of immunocomplexes, which triggers complement activation and opsonization of the immunocomplex by C3b and C3d molecules, which can target the immunocomplex to follicular dendritic cells and B cells via CD21 and CD35. Further, FcγR receptor mediated phagocytosis of IgG immunocomplexes by DCs is a very efficient mechanism of antigen uptake and processing.

In some embodiments, the antigens (e.g., TAA or VA) are carried on the surface of a cell, such as a tumor cell or a virally infected cell, along with the L-Rhamnose monosaccharide or Forssman disaccharide. Alternatively, the L-Rhamnose monosaccharide or Forssman disaccharide are conjugated to one or more isolated or synthetic TAA or VA through a chemical linker Exemplary bifunctional linkers include, NHS-activated linkers, which may include one or a combination of alkyl, ether, polyether, and/or polyamide groups in a spacer region.

In another aspect, the invention provides a method for inducing an immune mediated destruction of a vaccine target, such as a tumor cell or a virus-infected cell, in an animal. According to this aspect, the method comprises administering to an animal in need thereof, a composition described herein. In certain embodiments, the invention involves the use of tumor or virus-infected cells or isolated or synthetic TAA or VA as the immunogenic component. Where tumor cells are employed, the vaccine tumor cells may be allogeneic, syngeneic, or autologous.

Tumors which may be treated in accordance with the present invention include malignant and non-malignant tumors.

Viral infections that may be treated in accordance with the present inventions include but are not limited to human immunodeficiency virus (HIV-1 and HIV-2), influenza, hepatitis B (HBV), hepatitis C (HCV), herpes simplex virus (HSV-1), human papilloma virus (HPV).

### DESCRIPTION OF THE FIGURES

FIG. 1 shows (a) carbohydrate antigen/antibody-mediated vaccine with enhanced immunogenicity; and b) structures of α-Gal, Rha and Forssman disaccharide.

FIG. 2 shows evaluations of anti-Gal and anti-Rha antibodies. (a) ELISA assays of antibodies in pooled complement normal human serum. (b) ELISA assays of antibodies in wildtype mice serum. (c) Competitive ELISA assays of 8 common monosaccharides performed with pooled complement normal human serum. Rha conjugated BSA was used as immobilizing antigen, and free D-mannose (Man), D-glucose (Glc), N-Acetyl-D-glucosamine (GlcNAc), D-xylose (Xyl), L-fucose (Fuc), N-Acetyl-D-galactosamine (GalNAc), D-galactose (Gal) and L-rhamnose (Rha) were used as competing antigens (2-fold dilutions from 200 mM to 12.5 mM).

FIG. 3 shows the synthesis of two different NHS activated Rha linkers R1 and R2 (Scheme 1).

FIG. 4 shows the synthesis of corresponding NHS activated linkers without Rha moiety (Scheme 2).

FIG. 5 shows MALDI spectra of Rha-Linker conjugated proteins. (a) Conjugations with BSA (BSA, red; BSA-R1, green; BSA-R2, purple; BSA-Linker-1, dark blue; BSA-Linker-2, light blue). (b) Conjugations with OVA (OVA2+ was observed as major peaks from MALDI; OVA, red; OVA-R1, blue; OVA-R2, purple; OVA-Linker-1, dark green; OVA-Linker-2, light green).

FIG. 6 shows evaluation of anti-Rha IgG antibody after immunization, by ELISA assay. Five mice were immunized by OVA-R2 for 3 immunization periods (2 weeks/period). Comparison between BSA-R1 coating (with Rha) and BSA-Linker-1 coating (without Rha) specifically illustrate anti-Rha antibody by excluding other factors from both protein and linker portions.

FIG. 7 shows evaluations of anti-Rha IgG titers in four different groups of mice (BSA-R1 was used as coating protein). Group I: immunized with OVA-R2 plus adjuvant (OVA-R2+ adjuvant); Group II: immunized with OVA-linker-2 plus adjuvant (OVA-Linker-2+ adjuvant); Group III immunized with PBS plus adjuvant (PBS+adjuvant); Group IV: no treatment (none).

### DETAILED DESCRIPTION OF THE INVENTION

The present invention provides an immunogenic composition comprising a T-cell antigen in association with a Rhamnose monosaccharide (e.g., L-Rhamnose) and/or Forssman disaccharide, and provides corresponding methods for inducing immune response in an animal. Non-limiting examples of T-cell antigens include proteins of viral origin or proteins expressed by tumors. The invention takes advantage of the naturally high titers of anti-Rhamnose and/or anti-Forssman disaccharide antibodies in humans to target vaccine compositions to antigen presenting cells for effective processing and presentation to the immune system.

For example, in various embodiments the compositions and methods of the invention employ attenuated vaccine tumor cells, on which L-Rhamnose monosaccharide and/or Forssman disaccharide are present. The vaccine tumor cells may be attenuated, such as by gamma irradiation. The use of tumor cells in the general manner described herein but with α-Gal epitopes is described in WO 2004/032865, which is hereby incorporated by reference. In some embodiments, the Rhamnose or Forssman epitope is conjugated to the tumor cells, viral-infected cells, virus-like-particles (VLPs) or tumor associated antigen(s) (TAA) or viral antigens (VA) via a chemical linker. The chemical linker can facilitate intracellular processing of the antigen for effective presentation on APCs.

The compositions and methods of the invention may employ any antigen in association with the L-Rhamnose or Forssman disaccharide. The term “antigen” means any biological molecule (protein, peptide, lipid, glycan, glycoprotein, glycolipid, etc) that is capable of eliciting an immune response against itself or portions thereof, including but not limited to, tumor associated antigens and viral, bacterial, parasitic and fungal antigens. Generally, the antigen will have a peptide component for presentation by major histocompatibility complex class I or II. “Antigen presentation” means the biological mechanism by which macrophages, dendritic cells, B cells and other types of antigen presenting cells process internal or external antigens into subfragments of those molecules and present them complexed with class I or class II major histocompatibility complex or CD1 molecules on the surface of the cell. This process leads to growth stimulation of other types of cells of the immune system (such as CD4(+), CD8(+), B and NK cells), which are able to specifically recognize those complexes and mediate an immune response against those antigens or cells displaying those antigens.

In some embodiments, the composition comprises an autologous antigen, such as a cancer or tumor cell, for generating an immune response in a subject. The term “tumor cell” means a cell which is a component of a tumor in an animal (e.g., a malignant epithelial cell), or a cell that is determined to be destined to become a component of a tumor, i.e., a cell which is a component of a precancerous lesion in an animal. Included within the concept of the invention is the use of malignant cells of the hematopoietic system which do not form solid tumors such as leukemias, lymphomas and myelomas. The term “tumor” is defined as one or more tumor cells capable of forming an invasive mass that can progressively displace or destroy normal tissues.

The tumor or cancer cell carries one or more tumor associated antigens for generating an immune response, and carries one or more L-Rhamnose monosaccharide or Forssman disaccharide epitopes, optionally though a chemical linker. The term “Tumor Associated Antigens” or “TAA” refers to any protein or peptide expressed by tumor cells that is able to elicit an immune response in a subject, either spontaneously or after vaccination. TAAs comprise several classes of antigens: 1) Class I HLA-restricted cancer testis antigens which are expressed normally in the testis or in some tumors but not in normal tissues, including but not limited to antigens from the MAGE, BAGE, GAGE, NY-ESO and BORIS families; 2) Class I HLA restricted differentiation antigens, including but not limited to melanocyte differentiation antigens such as MART-1, gp100, PSA, Tyrosinase, TRP-1 and TRP-2; 3) Class I HLA restricted widely expressed antigens, which are antigens expressed both in normal and tumor tissue though at different levels or altered translation products, including but not limited to CEA, HER2/neu, hTERT, MUC1, MUC2 and WT1; 4) Class I HLA restricted tumor specific antigens which are unique antigens that arise from mutations of normal genes including but not limited to β-catenin, α-fetoprotein, MUM, RAGE, SART, etc; 5) Class II HLA restricted antigens, which are antigens from the previous classes that are able to stimulate CD4+ T cell responses, including but not limited to member of the families of melanocyte differentiation antigens such as gp100, MAGE, MART, MUC, NY-ESO, PSA, Tyrosinase; and 6) Fusion proteins, which are proteins created by chromosomal rearrangements such as deletions, translocations, inversions or duplications that result in a new protein expressed exclusively by the tumor cells, such as Bcr-Abl.

Viral antigens include any protein, glycoprotein or peptide expressed by a cell infected by a virus, and includes any proteins encoded by the virus DNA or RNA genome, that forms part of the viral core, matrix, envelope, nucleoprotein, RNA or DNA polymerases, integrases, or accessory regulatory proteins.

TAA or VA peptides include amino acid sequences of 5, 6, 7, 8, 9, 10, 11, 12, 13, 14, 15, 16, 17, 18, 19 or 20 amino acids that bind to MHC (or HLA) class I or class II molecules, and that have at least 70% amino acid identity with an amino acid sequence contained within the corresponding TAA or VA. Peptide sequences which have been optimized for enhanced binding to certain allelic variants of MHC class I or class II are also included within this class of peptides.

In some embodiments the cell carrying the antigen(s) and saccharide epitope is a xenogeneic cell. The term “xenogeneic cell” refers to a cell that derives from a different animal species than the animal species that becomes the recipient of the vaccination procedure. In other embodiments, the cell carrying the antigen(s) and saccharide epitope is an allogeneic cell. The term “allogeneic cell” refers to a cell that is of the same animal species but genetically different in one or more genetic loci as the animal that becomes the recipient. This usually applies to cells transplanted from one animal to another non-identical animal of the same species. In still other embodiments, the cell carrying the antigen(s) and saccharide epitope may be a syngeneic cell. The term “syngeneic cell” refers to a cell which is of the same animal species and has the same genetic composition for most genotypic and phenotypic markers as the animal who becomes the recipient host of the vaccination procedure.

The basic rationale for immune therapy against tumors is the induction of an effective and specific immune response against tumor-associated antigens (TAA), which in turn results in immune-mediated destruction of proliferating tumor cells expressing these antigens. Without intending to be bound by theory, a general description of the process, including certain embodiments, is as follows.

Tumor cells or virus-infected cells express antigens that can be recognized by the host's immune system. Endogenous TAA or viral proteins are degraded in the proteasome into 8-11 amino acid peptides which bind to the MHC class I. Each allelic MHC variant binds only a subset of peptides that share conserved amino acid residues at each position. The peptide-MHC complex is recognized by the T cell receptor (TCR) on the surface of T lymphocytes. Therefore, an exquisite level of specificity is achieved by presentation of certain peptides in the context of specific MHC classes and allelic variants that are recognized only by certain TCR molecules.

Effective prophylactic or therapeutic vaccines based on TAA or VA proteins or peptides have several requirements. First, the epitopes present in the vaccine have to be present in TAAs expressed by the tumor or in VA expressed in virus-infected cells. Second, the epitopes have to be effectively presented in the context of the right MHC alleles of the patient. Third, the vaccinating antigens must be properly captured, processed and presented by antigen presenting cells (APC) such as macrophages, dendritic cells and B cells. Within APCs, TAAs or VA are degraded in the lysosomal compartment and the resulting peptides are expressed on the surface of the APC membrane mostly in association with MHC Class II molecules and also in association with MHC class I molecules if the antigen traverses the cross-presentation pathway. This expression mediates recognition by specific CD4+ helper T cells or CD8+ effector T cells and subsequent activation of these cells to effect the immune response.

Most tumor cells have unique expression profiles of TAA, and in many cases the immunogenic peptides include a mutated amino acid sequence that confers immunogenicity through the exposure of an altered nonself epitope. These epitopes are usually very immunogenic. However, many tumors escape immune surveillance either 1) by not-generating these epitopes during proteasome processing, or 2) by down regulating the expression of MHC components such as β-microglobulin, or 3) because the immune system does not recognize these TAA as foreign antigens because either they are not presented in the context of a cellular “danger” signal, or 4) because the immune system has been tolerized to those antigens and recognizes them as “self” antigens. Immunotherapeutic approaches based on T-cell recognition of TAA-derived peptides are conventionally not expected to work using any vaccination approach for the two first cases (i.e. when the tumor does not present the antigenic TAA), but are considered well suited for the last two cases (i.e. when the immune system does not recognize the TAA as immunogenic).

One of the reasons for the lack of a sufficient immune response to control cancer growth in vivo is due to the poor immunogenicity of natural epitopes expressed by tumor cells. With the exception of the immunodominant melanoma Melan-A/MART127-35 and gp100 peptides, which readily activate specific T cells in vitro and in vivo, most T-cell responses require repeated in vitro stimulation with TAA epitopes and show limited immunogenicity when used as vaccines for cancer patients. In addition, vaccination with peptides may induce epitope specific T-cell tolerization rather than activation, depending on, for example, the adjuvant used and route of immunization.

One possible explanation for the limited therapeutic efficacy of TAA peptide vaccination lies in the fact that activation of peptide-specific CTL responses requires the delivery of inflammatory signals from monocytes, lymphocytes, or granulocytes recruited at the site of vaccination. Those signals may or may not be provided by standard adjuvants like incomplete Freund's adjuvant. An efficient activation signal, however, may be provided by natural adjuvants that trigger a “danger” signal such as bacterial DNA or synthetic oligodeoxynucleotides (ODN) containing unmethylated CpG dinucleotides (CpG-ODN). Such signals can stimulate B cells, natural killer (NK) cells, T cells, monocytes, and antigen-presenting cells; more importantly, such signals can promote maturation of DCs, a step that will result in the activation of the antibody and cell-mediated immune responses. More recently, CpG-ODN have been shown to improve the antitumor activity of antigen-presenting cells loaded with TAA peptides and promote a 10-fold to 100-fold increase in the induction of CTL responses to peptide immunization (Brunner et al. 2000).

The invention is based in part on the concept that antibodies binding to TAA or VA promote the formation of immunocomplexes, which bind to the FcγR receptors on APCs. Fc receptor targeting accomplishes several important functions for effective vaccine performance including promoting the efficient uptake of antigen for both MHC Class I and II antigenic presentation; promoting APC activation and maturation of dendritic cells. APCs that ingest a tumor cell must be activated before they can effectively present antigen. Otherwise, presenting antigens to immature APCs, without the required activation signals, can suppress the immune response. Further, the uptake of opsonized TAA, VA, or TAA-expressing cells by antigen presenting cells via FcγR receptor mediated endocytosis may be critical to generating an effective anti-tumor CTL response since it promotes the activation of MHC class I restricted responses by CD8+ T-cells through a cross-presentation pathway. Vaccines that cannot stimulate a humoral immune response are limited in their ability to induce cellular immunity by HLA restriction. CTLs are HLA restricted and will only destroy the tumor or virus-infected cells that present TAAs or VAs on self-class I MHC molecules. On the contrary NK cells will destroy the tumor vaccine cells if they are opsonized by antibodies by antibody-dependent cell cytotoxicity (ADCC).

More specifically, the different antigen uptake and processing pathways control the presentation of antigenic peptides by either MHC class I molecules to CD8+ T cells (endogenous pathway) or MHC class II molecules to CD4+ T cells (exogenous pathway). Vaccines that are composed of exogenous antigens use mainly the exogenous pathway for the delivery of antigen to APCs. This, in turn, favors the stimulation of CD4+ T cells and the production of antibodies. To deliver exogenous antigens to the endogenous pathway in order to elicit a cellular mediated response, the engagement of the FcγR receptor to mediate antigen uptake of immunocomplexes is very important as it stimulates the cross-presentation pathway (Heath and Carbone 2001). Studies indicate that, in addition to classical CD4+ priming, antigen acquired through endocytosis by DC through FcγR results in the induction of T cell effector immunity resulting in TH1 and class I restricted CD8+ T cell priming. Furthermore, engagement of FcγR also induces DC activation and maturation. Thus, the existing evidence indicates that antigenic targeting to FcγR on DC accomplishes several important aspects of T cell priming important for induction of an immune response: facilitated uptake of antigen, class I and class II antigen presentation and induction of DC activation and maturation.

With respect to the present invention, three mechanisms of antigen uptake are expected to take place. First, the exogenous pathway involving phagocytosis/pinocytosis that sends the antigens through the endosomal/lysosomal pathway which results in presentation of the processed antigen in the context of MHC class II surface molecules that activate the proliferation of CD4+ helper T cells. Second, FcγR -mediated antigen uptake of immunocomplexes involving anti-Rham or anti-Forssman antibodies will favor the cross-presentation pathway, resulting in antigen presentation in the context of MHC class I molecules, which will preferentially activate CD8+ cytotoxic T cells. Third, binding of TAA or VA molecules to membrane IgM present in naïve B-cells will result in B-cell activation and differentiation, and also in MHC class II antigen presentation that further stimulates proliferation of memory CD4+ T-cells that recognize those antigens. After activation and stimulation B-cells proliferate, differentiate and produce antibodies which bind to surface TAA or VA molecules present on the target tumor or virus-infected cells, facilitating killing of the cell by complement-mediated cell lysis, antibody dependent cell cytotoxicity and FcγR-dependent phagocytosis. Also, target cell destruction is mainly achieved by cytotoxic CD8+ T cells previously activated by differentiated dendritic cells and helper CD4+ T cells. In summary, a main advantage of the vaccines of the present invention over previous TAA or VA protein or peptide vaccines is that it achieves the in vivo formation of immunocomplexes in the absence of adjuvant. This leads to recruitment of antigen presenting cells, increased FcγR-mediated phagocytosis and antigen uptake that result in activation of both cellular and humoral branches of the immune response. The stronger initial immune reaction is expected to induce both a more effective immunity and the generation of a larger pool of memory cells. Therefore, taking advantage of the strong innate immune response to L-Rhamose-containing proteins establishes a firm basis for novel antitumor and antiviral immunotherapies.

The present invention therefore provides methods and composition for protein or peptide vaccines that contemplate the aspects mentioned above and overcome some of the current limitations associated with the development of vaccine, including but not limited to TAA or VA protein or peptide vaccines.

In the present invention, T-Cell antigens (e.g., TAA or VA) are modified by chemical functionalization with a L-Rhamnose monosaccharide or Forssman disaccharide epitope which promotes the in vivo formation of immunocomplexes with natural anti-Rham or anti-Forssman antibodies.

The binding of natural anti-Rham or anti-Forssman IgG or IgM to the saccharide epitopes present in the immunizing molecule facilitates the formation of immunocomplexes, which triggers complement activation and opsonization of the immunocomplex by C3b and C3d molecules, which can target the immunocomplex to follicular dendritic cells and B cells via CD21 and CD35, thereby augmenting the immune response. Also, FcγR receptor mediated phagocytosis of IgG immunocomplexes by DCs is a very efficient mechanism of antigen uptake and processing. Second, complement-activation at the site of vaccination generates a “danger signal” which has numerous implications for the kind of immune response that will be generated. Danger signals are recognized as crucial components for APC activation and differentiation to mature DCs. Additionally, complement activation has chemo-attractant properties that, similarly to GM-CSF, result in inflammation and recruitment of APCs.

Theoretically, there is no limitation in the identity or properties of the TAA or VA used for vaccination. A vast list of TAA has been compiled by Renkvist et al. (Novellino et al. 2005; Renkvist et al. 2001). All the TAA antigens cited in these publications are suitable for the method and compositions of the present invention and are incorporated herein by reference. Similarly, portions of the full length TAA amino acid sequences or their isoforms are well suited for the purposes of antitumor vaccination described in this invention. Exemplary TAA for association with L-Rhamnose monosaccharide or Forssman disaccharide in accordance with the invention are disclosed in US 2012-0003251, which is hereby incorporated by reference in its entirety.

In some embodiments, the tumor antigens or TAA are carried on a cell, such as a tumor cell, along with the L-Rhamnose monosaccharide or Forssman disaccharide. Methods for presenting the saccharide antigen on the surface of the cell are generally disclosed in WO 2004/032865, which is hereby incorporated by reference in its entirety. The L-Rhamnose monosaccharide or Forssman disaccharide are conjugated to one or more tumor cell components or isolated or synthetic tumor antigens through a chemical linker. Methods for chemical conjugation are well known. See, Mädler et al., Chemical cross-linking with NHS esters: a systematic study on amino acid reactivities, J Mass Spectrom. 2009 May;44(5):694-706. Methods and exemplary chemical linkers are disclosed in US 2012-003251, which is hereby incorporated by reference in its entirety.

Exemplary bifunctional linkers include N-hydroxy-succinimide-L1-Maleimide cross-linker (NHS-R-Mal, where L1 is any type of linear linker such as but not limited to: alkyl, ether, polyether, polyamide, and combinations thereof). A maleimide activated L-Rham or Forssman molecule can be reacted to Cysteine residues in a purified TAA or VA protein, thereby yielding a L-Rham/Forssman (+) TAA or VA protein.

Alternatively, saccharide epitopes having a primary amino group can be enzymatically coupled to the γ-carboxamide residue of glutamine by bacterial glutaminyl-peptide γ-glutamyl transferase (Transglutaminase). Reduction of thioethyl group to sulphydryl group leaves a free-SH group that is reactive with Maleimide-R2-NHS linkers. This activated epitope can be coupled to proteins or peptides bearing primary amino groups either at the N-terminus or at lysine residues. Similarly, bifunctional NHS-R1-NHS linkers could be coupled to the free NH₂ group of L-Rham or Forssman molecules and then coupled to the ε-NH₂ group of lysines present in the TAA or VA protein or peptides.

The above mentioned Rham or Forssman epitopes could also be used to modify synthetic peptides that bear amino acids such as Cysteine, Homocysteine, Serine, Threonine or Glutamine, by post-synthesis chemical conjugation of the activated L-Rham epitope to the pure synthetic peptide in the same way as described for TAA or VA proteins.

In the present invention, the purpose of modification of peptides or proteins with L-Rham or Forssman epitopes is to mediate the in vivo formation of immunocomplexes with natural antibodies, thereby facilitating FcγR-mediated uptake of the immunocomplex, which will ultimately lead to enhanced presentation of the deglycosylated immunogenic epitopes, thereby triggering immunity. Therefore, some considerations regarding processing and presentation of glycosylated antigens are important to take into account when performing chemical modification of proteins or peptides. Glycoprotein antigens are ingested by APCs by endocytosis and transported from the cell surface toward the lysosomal compartments. During transport, proteolytic enzymes become activated as the pH of the endosome decreases. The enzymes, which include endoproteases and exoproteases with many different substrate specificities, attack and fragment the antigen into peptides. Glycans in a glycoprotein or glycopeptide can interfere with the proteolytic fragmentation and influence the pattern of T cell epitopes that are presented. Appropriate peptides (8-15 amino acids) are protected against further proteolysis as they bind to empty MHC class II molecules that are accumulating within the acidic compartments. Finally, the MHC-peptide complexes are transported to the cell surface and presented to CD4+ T cells. Due to the fact that many cellular proteins are extensively glycosylated, processing and presentation mechanisms are expected to produce a pool of major MHC−0 bound protein-derived peptides, part of which retain sugar moieties. It has been demonstrated that T cells are able to recognize partially glycosylated peptides that bind to the MHC molecules if the sugar moiety is small and if it is located in a central position within the peptide being presented. Sugar moieties present at the ends of the peptide being presented do not elicit an immune response against the glycosylated portion of the peptide. In the present invention, the objective is to elicit an immune response against deglycosylated peptides or against the non-glycosylated portion of the glycopeptides, since the target TAAs or VA expressed by tumors or virus-infected cells do not bear the same glycosydic modification as the immunizing peptides. Since the chemical addition of L-Rham or

Forssman epitopes mediated by N-hydroxysuccinimide, Maleimide or other functional groups will not create the natural N-linked chemical bonds of sugar to Asparagine residues, or the natural O-linked sugar moieties to Serine or Threonine residues, complete removal of sugar moieties (that do not contain natural N-linked or O-linked chemical bonds) is anticipated to be impaired during antigen processing.

In some embodiments, removal of the L-Rham or Forssman epitope bound to a peptide or protein during antigen processing and presentation can be facilitated by including one or more groups in the linker bridging the trissaccharide portion of the L-Rham or Forssman epitope with the peptide or protein. These groups have to be sensitive to cleavage by endocellular proteins such as esterases, peptidases, sulfhydryl reductases or glycosidases. After endocytosis, the enzymes of different specificities cleave the L-Rham or Forssman epitope at the ester group present in the linker region, thereby yielding a deglycosylated peptide that can bind to the MHC class I and II and elicit an immune response by engaging with TCR present in CD4+ and CD8+ T cells.

An alternative embodiment to prevent potential difficulties associated with incomplete deglycosylation of immunizing glycopeptides is to separate the region of the peptide known to trigger an immune response against cells expressing the corresponding TAA or VA from the region conjugated to the L-Rham or Forssman epitope. This can be done by creating an L-Rham or Forssman disaccharide tag fused to the immunogenic peptide. The tag consists of a stretch of 1 to 20 amino acids that bear the amino acids to which the saccharide epitope will be covalently linked, in addition to known endoprotease amino acid consensus sequences that will facilitate its cleavage by endosomal proteases. In this manner, the saccharide tag will mediate formation of immunocomplex with anti-Rham or anti-Forssman disaccharide antibodies, thereby enhancing DC activation, antigen processing and presentation. The saccharide tag will be released from the immunogenic portion of the peptide by proteases and aminopeptidases during antigen processing. The release of the non-glycosylated immunogenic portion of the peptide is expected to bind to the MHC-II complex or the MHC-I complex in case of cross-presentation.

In certain embodiments, the chemical addition of L-Rham monosaccharide or Forssman disaccharide epitopes is performed on amino acid residues corresponding to a “tag” region adjacent to the amino acid sequence derived from the TAA or VA. In another embodiment, chemical addition of the L-Rham or Forssman epitope is performed to the N-terminal and/or C-terminal amino acid of the immunizing peptide.

For the in vivo formation of immunocomplexes capable of complement activation, each C1 molecule must bind to at least two Fc sites for a stable C1-antibody interaction. Circulating IgM exists in a planar configuration and does not expose the Clq binding sites. IgM exposes its C1q binding sites after binding to an antigen on a membrane. For this reason immunocomplexes formed by anti-Rham (or anti-Forssman) IgM and soluble L-Rham(+) TAA/VA or Forssman(+) TAA/VA will not likely activate the complement cascade. On the contrary, an IgG molecule contains only a single C1q binding site in the CH2 domain of the Fc portion of the immunoglobulin, so that stable C1q binding is achieved only when two IgG molecules are within 30-40 nm of each other in a complex, thereby providing two C1q binding sites. In order to form particulate immunocomplexes containing more than one IgG and one Rham/Forssman(+) TAA/VA molecule, each TAA/VA molecule has to contain more than a single saccharide epitope. This is easily achievable for proteins that have been chemically modified with L-Rham or Forssman epitopes at their lysine and/or cysteine residues. However, it is important to provide amino acids that serve as anchoring points for the chemical addition of L-Rham or Forssman epitopes and that do not form part of the immunogenic portion of the peptide.

Therefore, in another embodiment, L-Rham or Forssman disaccharide epitopes are chemically added in vitro to synthetic peptides with a structure comprising: 1) a sequence of 1-20 amino acids at its amino terminus that contains the acceptor amino acids for the L-Rham epitopes, 2) a central 7-20 amino acid sequence of a TAA or VA epitope known to elicit an immunogenic CD4+ or CD8+ T cell response, and 3) a sequence of 1-20 amino acids at the C-terminus that contains acceptor amino acids for addition of a second L-Rham epitope.

In an exemplary synthesis method the epitope is directly linked to the amino acid with no other glycosidic residues between the two, and the linkage will depend on the type of cross-linker used, such as maleimide where the epitope is added to cysteines, or succinimide where it will be bound to lysine and the primary N-terminal amino group, or glutaraldehyde where it will bind to serine or threonine. These different epitope linkages may provide certain distinct advantages in binding capacity of anti-Rhamnose and anti-Forssman antibodies and their capacity to form immunocomplexes.

In some embodiments, the cell or purified or synthetic antigen has one or more additional epitopes to target the composition to APCs, including for example, an α-galactosyl epitope. α-Gal epitopes are described, for example, in WO 2004/032865, which is hereby incorporated by reference in its entirety. Such additional epitopes may be conjugated separately to the same or different cells or purified or synthetic antigens. In some embodiments, the various saccharides for targeting the composition to APCs are combined into a single saccharide chain.

According to the invention, purified TAA or VA proteins, protein fragments or peptides modified to contain L-Rham or Forssman disaccharide epitopes are used as either prophylactic or therapeutic vaccines to treat tumors. Thus the invention also includes pharmaceutical preparations for humans and animals comprising L-Rham or Forssman(+) TAA or VA proteins or peptides. Those skilled in the medical arts will readily appreciate that the doses and schedules of pharmaceutical composition will vary depending on the age, health, sex, size and weight of the human and animal. These parameters can be determined for each system by well-established procedures and analysis e.g., in phase I, II and III clinical trials and by review of the examples provided herein.

In yet another related aspect, the invention provides a method for inducing an immune mediated destruction of tumor cells or virus-infected cells in an animal. According to this aspect, the method comprises administering to an animal in need thereof, a composition described herein. In still another aspect, the invention provides a method for treating an animal with tumor cells. The method comprises administering to the animal a therapeutically effective amount of a composition described. In certain embodiments, the invention involves the use of tumor cells as the immunogenic component. The vaccine tumor cells may be allogeneic, syngeneic, or autologous. In some embodiments, the invention employs a plurality of autologous tumor cells, which may be the same or different, for vaccination. The autologous tumor cells may be administered separately or together.

The compositions of the invention are generally administered in therapeutically effective amounts. The term “therapeutically effective amount” as to tumor treatment means an amount of treatment composition sufficient to elicit a measurable decrease in the number, quality or replication of previously existing tumor cells as measurable by techniques including but not limited to those described herein.

Tumors which may be treated in accordance with the present invention include malignant and non-malignant tumors. Malignant (including primary and metastatic) tumors which may be treated include, but are not limited to, those occurring in the adrenal glands; bladder; bone; breast; cervix; endocrine glands (including thyroid glands, the pituitary gland, and the pancreas); colon; rectum; heart; hematopoietic tissue; kidney; liver; lung; muscle; nervous system; brain; eye; oral cavity; pharynx; larynx; ovaries; penis; prostate; skin (including melanoma); testicles; thymus; and uterus. Examples of such tumors include apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), plasmacytoma, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing's experimental, Kaposi's, and mast-cell), neoplasms and for other such cells.

The term “treat” or “treating” with respect to tumors or cancer, refers to stopping the progression of the tumor or malignant cells, slowing down growth, inducing regression, or amelioration of symptoms associated with the presence of said cells.

For administration, the composition of the invention can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

Suitable formulations for parenteral, subcutaneous, intradermal, intramuscular, oral or intraperitoneal administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers. Also, compositions can be mixed with immune adjuvants well known in the art such as Freund's complete adjuvant, inorganic salts such as zinc chloride, calcium phosphate, aluminum hydroxide, aluminum phosphate, saponins, polymers, lipids or lipid fractions (Lipid A, monophosphoryl lipid A), modified oligonucleotides, etc.

In addition to administration with conventional carriers, active ingredients may be administered by a variety of specialized delivery drug techniques which are known to those of skill in the art.

The invention will now be described with respect to the following examples; however, the scope of the present invention is not intended to be limited thereby. All citations to patents and journal articles are hereby expressly incorporated by reference.

### EXAMPLES

In order to evaluate L-rhamnose antigen in pre-clinical applications, Rha-conjugated immunogens were synthesized, and as shown herein, successfully induced high titers of anti-Rha antibodies in wildtype mice. Moreover, the following examples demonstrate that wildtype mice could replace α1,3 galactosyltransferase knockout (α1,3GT KO) mice in such antigen/antibody mediated vaccine design when developing immunotherapies.

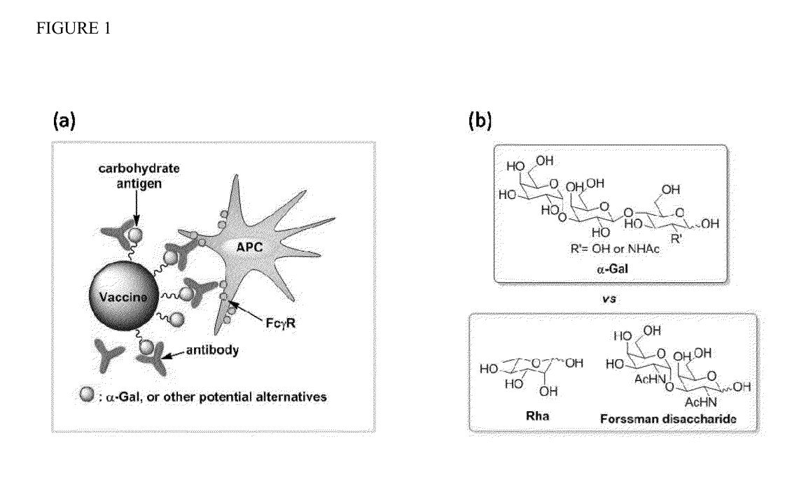

Gal-α(1,3)-Gal-β(3(1,4)-GlcNAc/Glc, termed the α-Gal epitope, represents one of the most well-known carbohydrate antigens, playing a crucial role in organ xenotransplantation. The significance of this unique antigen originates from the fact that anti α-Gal antibodies (anti-Gal) are naturally present in large amounts in humans, constituting about 1% of serum IgG (1-2). This aspect of the α-Gal epitope makes it an important target in potential clinical treatment. Besides its importance in xenotransplantation, the α-Gal epitope has been applied to enhance immunogenicity of vaccines by forming in vivo complexes with natural anti-Gal antibodies. Specifically, the injection of an α-Gal conjugated vaccine could result in in situ complexes of anti-Gal/α-Gal, thus effectively targeting the vaccine to antigen presenting cells (APCs) by the interaction between the Fc portion of the anti-Gal antibody and Fcγ receptors (FcγR) on APCs (FIG. 1a) (3-4). This promising feature of α-Gal in clinical medicine implies the similar potential of other carbohydrate antigens with naturally high antibody levels.

Several advanced high-throughput carbohydrate arrays have been successfully developed for evaluating carbohydrate-protein interactions (5-7). Further, comprehensive carbohydrate antigen arrays profiling human serum have been prepared (8,9). Consequently, other than anti-Gal antibodies, a number of anti-carbohydrate antibodies exist at relatively high levels. High levels of antibodies against mono-L-rhamnose (Rha) and GalNAc-α(1,3)-GalNAc (Forssman disaccharide) across all individuals (FIG. 1b) have been shown.

The following examples evaluate whether Rha or the Forssman disaccharide might be attractive alternatives to the α-Gal epitope for vaccine development. Early studies have immunologically characterized Rha bearing O-antigens from a variety of bacteria (10-13).

Rha oligosaccharides are constituents of carbohydrate units of microbial, immunogenic heteroglycans and lipopolysaccharides, in which they often function as the immuno-determinant groups of these immunogens (14). Rha was also isolated from Buckthorn (Rhamnus) (15) and poison sumac. In addition, Rha is a component of the outer cell membrane of acid-fast bacteria in the Mycobacterium genus, which includes the bacterium that causes tuberculosis. Unfortunately, the complexity of these identified Rha oligosaccharides restrained them from further study and application. The high levels of anti-Rha antibodies could offer advantages in the use of Rha antigen in potential therapeutics, due to its natural abundance and structural simplicity.

Our studies reveal that wildtype mice do not have high levels of anti-Rha antibodies. In order to establish an animal model for pre-clinical evaluations of the Rha antigen, the following examples provide a method for immunizing mice by synthetic Rha antigens for production of significant amounts of anti-Rha antibody titers, comparable to those of humans.

### RESULTS

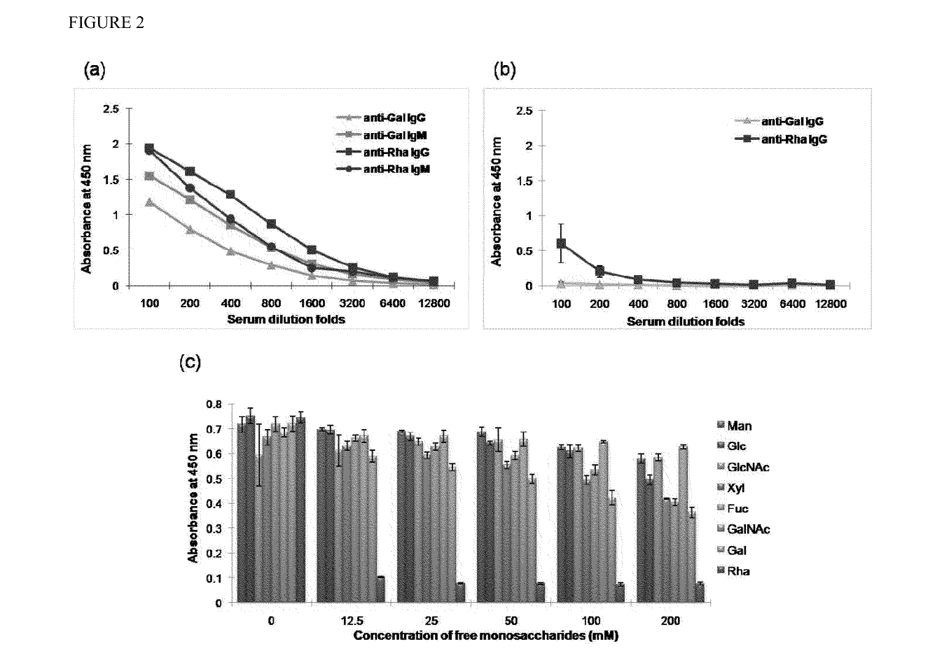

Pre-existing high anti-Gal titers in human serum have distinguished the α-Gal from other carbohydrate antigens in potential clinical applications over recent decades. In order to confirm the comparably high level of natural anti-Rha antibodies in human serum, ELISA experiments were performed to evaluate both titers of anti-Rha antibodies and anti-Gal antibodies. The expected high levels of anti-Gal IgG (1:1600) and IgM (1:3200) were confirmed in pooled normal human serum (FIG. 2a). Encouragingly, both high titers of anti-Rha IgG (1:6400) and IgM (1:6400) were also identified, in which anti-Rha IgG titers are four times higher than those of anti-Gal. In addition, a free monosaccharides competitive ELISA further validated high levels of anti-Rha titers against Rha in human serum (FIG. 2c). In this experiment, only the free Rha pulled down the antibodies in the human serum, but the other seven common monosaccharides did not. This indirectly indicates the existence of the antibodies specific to Rha. On the other hand, we examined the levels of anti-Gal and anti-Rha in wildtype mice serum (FIG. 2b). In contrast to humans, natural anti-Rha antibodies were observed at low pre-existing levels (1:800). Additionally, there was no evidence of anti-Gal existing naturally in mice and this antibody cannot be elicited in wild type mice. This suggested that wildtype mice could be used for evaluating Rha-associated cancer immunotherapies, whereas using α-Gal requires α1,3 galactosyltransferase knockout (α1,3GT KO) mice. These results suggest that Rha may be a promising alternative for α-Gal in preclinical animal experiments and clinical applications for humans.

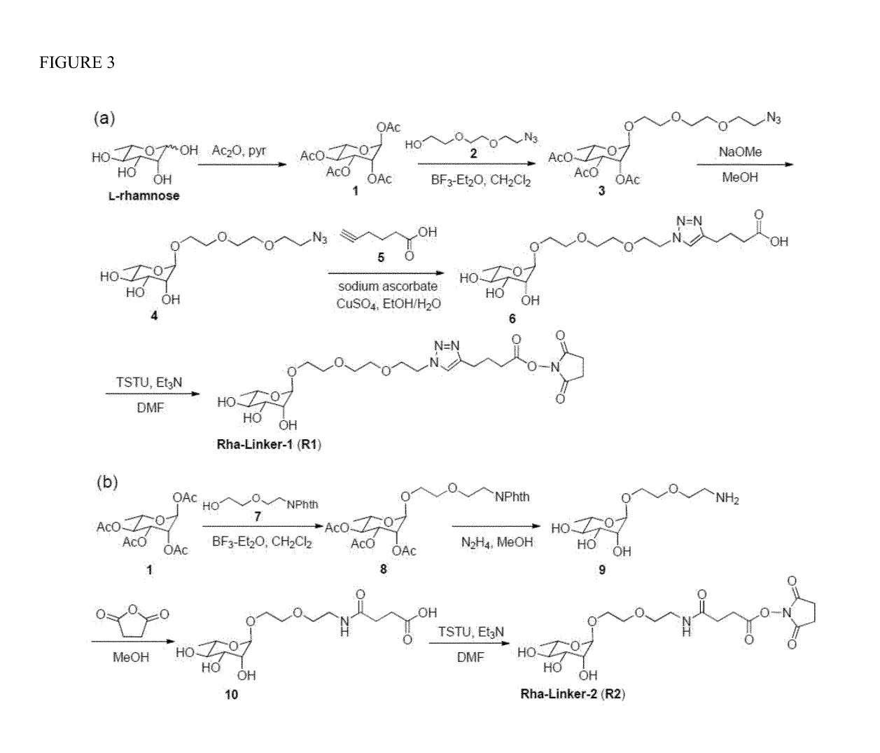

With successful validation of high titers of anti-Rha antibodies across human but not in wildtype mice, Rha conjugated protein antigens for immunization were designed and synthesized to establish an optimal mouse model with high anti-Rha antibody titers for future evaluation. The synthesis of a Rha antigen was divided into two steps: (1) installation and activation of a linker on Rha; and (2) chemical ligation between the Rha-linker and carrier protein. Rha was furnished with two different spacers bearing an N-hydroxysuccinimide (NHS) ester, which could readily conjugate with multiple lysine residues on carrier proteins under mild physiological condition (16). Syntheses of Rha-linker-1 (R1) and Rha-linker-2 (R2) are illustrated as Scheme 1 (FIG. 3), which furnished

Rha with two different spacers in order to avoid cross-interaction effects of linkers between immunization and ELISA assays. The synthesis of R1 started from the free L-rhamnose (Scheme 1a). Peracetylation of the starting material gave pure intermediate 1 with a configuration in quantitative yield without purification. The following glycosylation between peracetate donor 1 and azido linker 2 promoted by BF3-Et2O led to compound 3 with predominant a selectivity. Deacetylation of compound 3 by NaOMe resulted in azido linker 4. Subsequent installation of the carboxylic acid function group was accomplished by a copper-catalyzed Huisgen 1,3-dipolar cycloaddition (17) between compound 4 and the 5-hexynoic acid 5. Final conversion of the carboxylic acid to an NHS activated ester was initially performed through traditional method, by which NHS and N,N′-diisopropylcarbodiimide (DIC) were used. However, these attempts provided unsatisfactory activation results. Conversely, utilizing N,N,N′,N′-tetramethyl-O-(Nsuccinimidyl)-uronium tetrafluoroborate (TSTU) (18-19), an activated form of NHS, offered a much better activation of the acid 6 to furnish Rha-Linker-1 (R1) in anhydrous DMF solvent in the presence of Et₃N. By applying a minimal amount of each reagent during the activation step, the crude product, following solvent removal, was directly used for conjugation with carrier proteins, or stored in freezer for at least one year with intact reactivity. This activation strategy allowed for convenient preparation and storage of NHS activated linkers in a relative large scale.

The alternative synthesis of Rha linker employed the intermediate 1 (Scheme 1b, FIG. 3). The installation of phathalimide protected amine linker was achieved by using linker acceptor 7 (20) through glycosylation with peracetate precursor 1. Then all protecting groups on intermediate 8 were removed by treatment with hydrazine in anhydrous MeOH to give amine 9. The reaction between 9 and succinic anhydride in MeOH yielded regioselective amination product 10 with a terminal carboxylic acid group.

Finally, the acid was successfully activated by TSTU to generate the linker Rha-Linker-2 (R2).

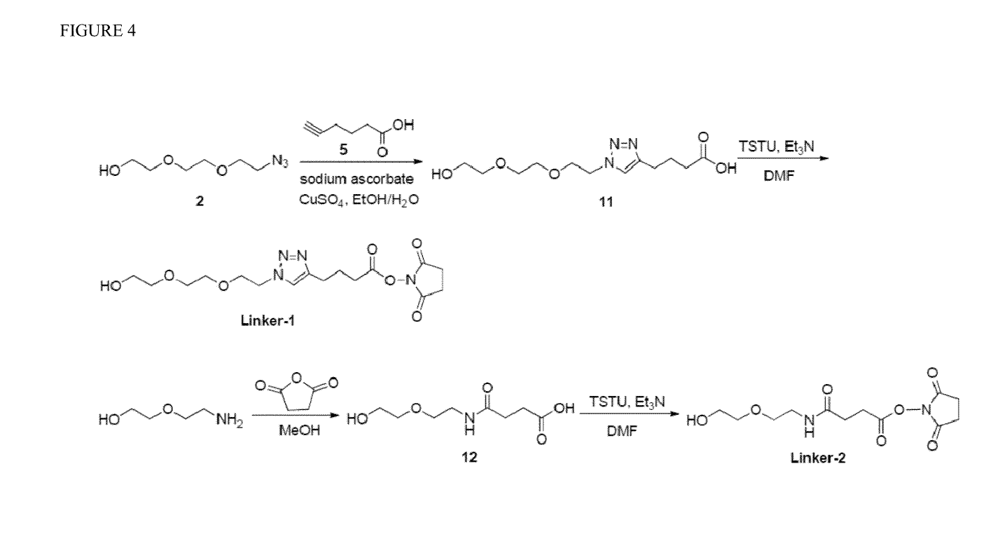

To specifically characterize the production of antibodies against the Rha epitope during the follow-up bioassay, two NHS linker counterparts were prepared, which were later conjugated with coating protein for ELISA assays. For this purpose, Linker-1 and Linker-2 were synthesized via intermediates 11 (21) and 12 (22), followed by the same activation procedures as for the two Rha linkers (Scheme 2, FIG. 4). This “linker-only” design allowed us to address any additional immunogenic effect from any component other than the Rha moiety on the synthetic glycoprotein antigens.

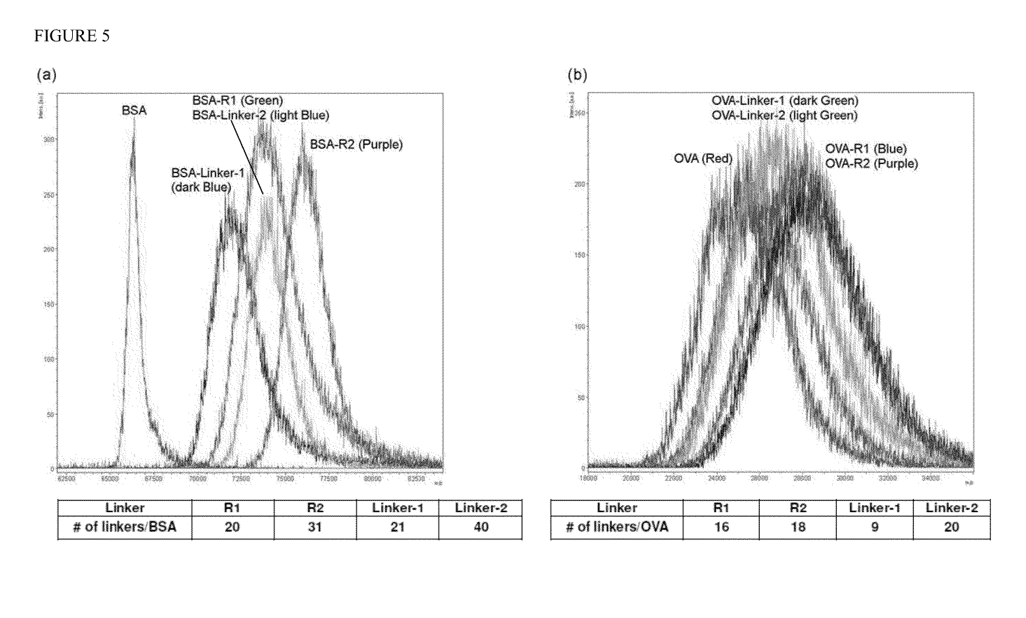

Following the synthesis of these NHS activated linkers, conjugations with carrier protein under different conditions were investigated in order to obtain optimal Rha immunogens. The initial model reactions employed bovine serum albumin (BSA) as a carrier protein in order for convenient characterization by SDS-PAGE and mass spectrometry. It was suggested that 3×PBS was the best medium for such conjugation presumably because that it provides better buffer capacity and more accessible sites for ligation. With this conclusion, all the following ligations were carried out in 3×PBS for 1 hour and quenched by ultrafiltration to remove the excess linkers. The conjugation results were characterized by both SDS-PAGE and MALDI to estimate the number of linkers per protein molecule. This strategy successfully led to Rha/Linker conjugated proteins in good yields (FIG. 5).

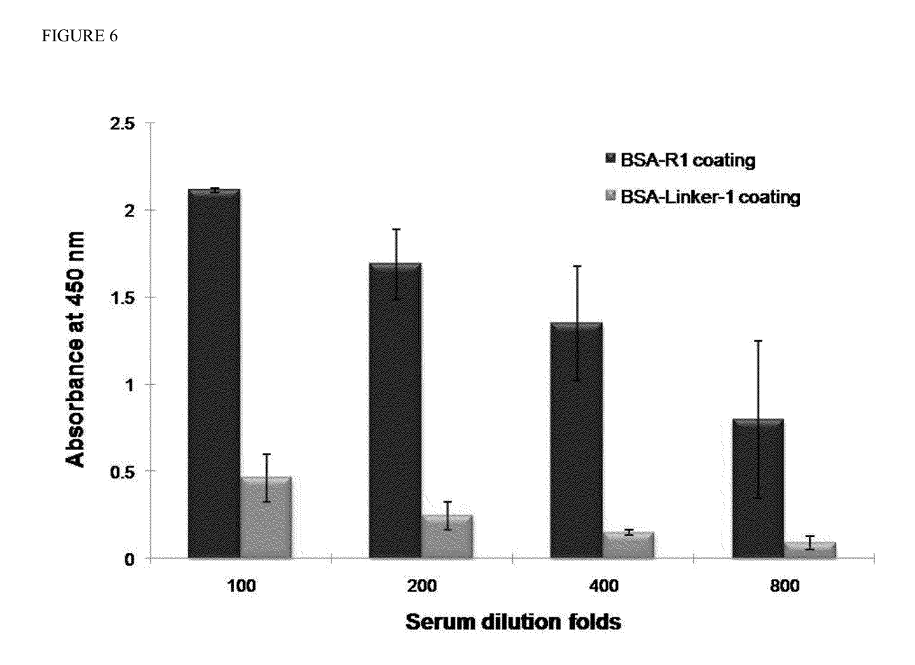

With these synthetic Rha-antigens ready, the first goal was to establish a protocol to produce high titers of anti-Rha antibodies in wildtype mice. The initial mice immunization and assay procedure used R2 conjugated BSA (BSA-R2) as an immunogen, and R1 conjugated ovalbumin (OVA-R1) was employed as coating antigen for ELISA assays. Unfortunately, this method failed to show unambiguously anti-Rha antibody production. After deliberated consideration of all possible factors, it appeared that OVA might not be a suitable coating protein for ELISA assays, in that the OVA is a glycoprotein. Consequently, its native glycan moiety might bind with non-Rha related antibodies in mouse serum, thus interfering with the ELISA assay. In order to address this concern, an alternative modified protocol using R2 conjugated OVA (OVA-R2) as immunogen and R1 conjugated BSA (BSA-R1) as coating protein was carried out. This modification was then able to show the distinctly different amounts of antibody IgG titers against two different coating antigens (BSA-R1 and BSA-Linker-1), which explicitly indicated the successful production of antibody specifically against the Rha monosaccharide in the immunized mice (FIG. 6). These dramatically different results also confirmed our early assumption of OVA interference during the assay. Some non-specific antibodies produced by Freund's complete adjuvant (FCA) (since the FCA contains heat-killed Mycobacterium tuberculosis, therefore, anti-bacteria carbohydrate antibodies may be also induced) might bind to the carbohydrate moieties on OVA when it was used for coating, and thus disturbed the previous ELISA assay.

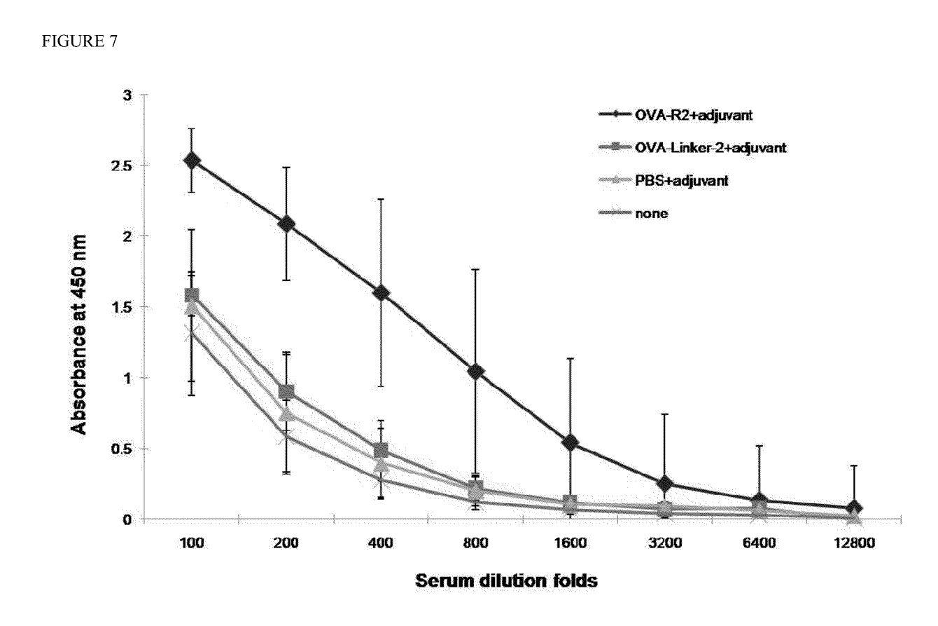

After achieving the successful production of the anti-Rha antibodies in wildtype mice, we moved forward to immunize an appreciable group of mice, which was divided into four sub-groups with a control. By using the established procedure, remarkable differences in anti-Rha antibody productions were observed as expected among these sub-groups after three immunization periods. The ELISA assays showed that only the mice in OVA-R2 immunization group produced significantly high titers of anti-Rha IgG antibody, while the other three control groups (corresponding to “OVA-Linker-2+adjuvant”, “PBS+adjuvant” and “none” treatment groups) maintained low levels of corresponding antibody (FIG. 7). It was very clear that ELISA assay by BSA-R1 coating could competently reflect the specific binding between its Rha moiety and the induced anti-Rha antibody (FIG. 6). Therefore, the titers of anti-Rha IgG could be directly calculated from the absorbance reading. Our results indicated that the titers of anti-Rha IgG after Rha-immunization reached 1:6400, while those of the other three groups were 1:1600, 1:1600 and 1:800, respectively. It is worth mentioning that the natural anti-Rha antibody in wildtype mice gradually increased by aging during the whole immunization period, but could never reach the same level as that in the immunized group. In addition, a few individual mice exhibited extreme sensitive or inert responses to the Rha immunization. Nevertheless, the overall trend of antibody level after immunization demonstrated the successful production of a wildtype mice model with high anti-Rha titers, which is as high as that in natural human serum.

The established anti-Rha mouse model, as well as the immunization procedure, should provide valuable knowledge for future cancer immunotherapies involving the Rha antigen.

We confirmed the existence of high titers of anti-Rha antibodies in humans, even at higher levels than anti-Gal antibodies. In addition, identification of naturally low titers of anti-Rha antibodies demonstrated the absence of natural Rha synthase in this model. Based on these pre-evaluations, Rha-conjugated immunogens have been designed and synthesized. To our knowledge, these immunization results presented the first successful production of high titers of anti-Rha antibodies in wildtype mice, which reached levels similar to those observed in humans. Furthermore, this study provides significant evidence that a single monosaccharide antigen is able to elicit B cell immunity for antibody production.

The targeting of autologous vaccines to APCs through the in vivo complexation of antigen/antibody presents a promising cancer immunotherapy with enhanced immunogenicity. This strategy relies on the ubiquitous presence of certain antibodies in human serum. Our studies suggest that the monosaccharide Rha could become a promising alternative in the development of cancer or antiviral immunotherapies, in that wildtype mice, as well as many of other non-primate animals, could be directly used for pre-clinical evaluations.

Methods General procedure for linker activation. TSTU (1.1 eq.) and Et3N (1.5 eq.) were added to a solution of acid linker (1 eq.) in anhydrous DMF. The reaction was monitored by LC-MS. After stirring at RT for 1 h, the free acid completely disappeared. The reaction mixture was then concentrated and dried under vacuum to give crude NHS activated linkers which were stored in 20° C., and directly used in the following conjugations without further purification.

General procedure for conjugation between linkers and proteins. The synthetic linkers in solution (10 mg mL−1 in 3×PBS) were added to the same volume of protein solution (10 mg mL−1 in 3×PBS) and was stirred at RT for 1 h. Then the resultant solution was ultrafilterated and washed with lx PBS using Amicon® Centrifugal Filter Devices (Ultracel® 10,000). The collected glycoprotein solution was quantitated by Pierce BCA Protein Assay Kit (Pierce) and stored at 4° C. for following immunological evaluation. The yields of the glycoproteins varied from 85% to 95% based on the colorimetric detection and quantification of total protein using this protocol. MALDI analysis of the glycoconjugates was performed by using Bruker Microflex TOF.

SDS-PAGE. Protein conjugates were suspended in 12 μL sample buffer (5% (w/v) SDS, 10% (v/v) glycerol, 25 mM Tris-C1, pH 6.8, 10 mM DTT, 0.01% (w/v) bromophenol blue), loaded on different lanes of a 1.5 mm-thick, 12% (w/v) SDS-PAGE gel, and visualized by Coomassie Brilliant Blue R-250 staining

ELISA assay for detecting anti-Rha antibodies. 96-well ELISA plates were coated at 4° C. overnight with coating protein (Rha conjugated protein) (10 μg mL−1) in 1×PBS buffer (pH 7.4). The plates were washed twice with PBS buffer containing 0.2% (v/v) Tween 20 (PBST), then blocked by 5% (w/v) non-fat milk in PBST at 4° C. overnight. The plates were washed and then incubated for 2.5 h at RT with human or mice sera in two-fold dilution with PBST from 1:100. The plates were washed three times with PBST, followed by the incubation with anti-human IgG or IgM specific horse radish peroxidase-conjugated antibodies (Invitrogen, USA) for 1.5 h at RT. After the plates were washed, enzyme substrate tetramethylbenzidine (TMB) was added and allowed to react for 10-20 min before the enzymatic reaction was terminated by adding 1N HCl and the absorbance was read at wavelength of 450 nm in FlexStation® 3 Microplate Reader (Molecular devices). The titers were calculated to the highest dilution that gave the OD value beyond 0.1.

ELISA assay for detecting anti-Gal antibody. 96-well ELISA plates were coated at 4° C. overnight with coating protein (α-Gal conjugated BSA) (10 μg mL₋₁) in 1×PBS buffer (pH 7.4). The remaining procedure followed the same one as for previous anti-Rha antibody assays.

Competitive ELISA assay. To further verify the specific antibody against Rha epitopes, inhibition ELISA was performed by immobilizing Rha conjugated BSA (BSA-R1) (10 μg ml⁻¹) on the 96-well plate, and free D-mannose (Man), D-glucose (Glc), N-Acetyl-Dglucosamine (GlcNAc), D-xylose (Xyl), L-fucose (Fuc), N-Acetyl-D-galactosamine (GalNAc), D-galactose (Gal) and L-rhamnose (Rha) were used as competing antigens (2-fold dilutions from 200 mM to 12.5 mM). After coated with BSA-R1 at 4° C. overnight, the solution was depleted and washed by PBST for three times (three minutes each time). Then, the plate was blocked with 5% (w/v) non-fat milk (PBST) in RT for 1.5 h, and rinsed by PBST once. Normal human serum diluted (1:2000) previously, containing different free monosaccharide at different concentration, was added into the 96-well plate with 0.1 mL per well. After 2 h incubation, the plate was rinsed by PBST 3 times. Then 0.1 mL Horseradish Peroxidase (HRP)-conjugated antihuman IgG antibody (1:3000) was added into each well and stayed in RT for 1 h. Finally, after the plates were washed, enzyme substrate tetramethylbenzidine (TMB) was added and allowed to react for 10-20 min before the enzymatic reaction was terminated by adding 1N HCl and the absorbance was read at wavelength of 450 nm in a FlexStation® 3 Microplate Reader (Molecular devices).

Mice and immunization procedures. The mice (female, BALB/c, 6-8 weeks) obtained from The Jackson Laboratory were maintained at the animal facility of The Ohio State University. Groups of at least 5 mice were immunized subcutaneously (several different sites with a total of 150 μL) on days 0, 14 and 28 with 30 μg of Rha conjugates. Freund's complete adjuvant (FCA), incomplete adjuvant (FIA) and no adjuvant were used respectively in the above 3 times of immunizations. The mice were bled on the 7th day after 3rd immunization (tail vein) and the sera were tested for the presence of anti-Rha antibodies. All experiments with mice were performed according to IACUC (Institutional animal care and use committee) guidelines.

### REFERENCES

1. Galili, U., Rachmilewitz, E. A., Peleg, A., and Flechner, I. (1984) A unique natural human IgG antibody with anti-alpha-galactosyl specificity, J. Exp. Med. 160, 1519-1531.
2. Macher, B. A., and Galili, U. (2008) The Galalphal,3Galbetal,4GlcNAc-R (alpha-Gal) epitope: a carbohydrate of unique evolution and clinical relevance, Biochim. Biophys. Acta 1780, 75-88.
3. Galili, U. (2005) The alpha-gal epitope and the anti-Gal antibody in xenotransplantation and in cancer immunotherapy, Immunol. Cell Biol. 83, 674-686.
4. Abdel-Motal, U. M., Wigglesworth, K., and Galili, U. (2009) Mechanism for increased immunogenicity of vaccines that form in vivo immune complexes with the natural anti-Gal antibody, Vaccine 27, 3072-3082.
5. Liang, P. H., Wu, C. Y., Greenberg, W. A., and Wong, C. H. (2008) Glycan arrays: biological and medical applications, Curr. Opin. Chem. Biol. 12, 86-92.
6. Lee, M. R., Park, S., and Shin, I. (2006) Protein microarrays to study carbohydrate-recognition events, Bioorg. Med. Chem. Lett. 16, 5132-5135.
7. Paulson, J. C., Blixt, O., and Collins, B. E. (2006) Sweet spots in functional glycomics, Nat. Chem. Biol. 2, 238-248.
8. Oyelaran, O., McShane, L. M., Dodd, L., and Gildersleeve, J. C. (2009) Profiling Human Serum Antibodies with a Carbohydrate Antigen Microarray, J. Proteome Res. 8, 4301-4310.
9. Huflejt, M. E., Vuskovic, M., Vasiliu, D., Xu, H., Obukhova, P., Shilova, N., Tuzikov, A., Galanina, O., Arun, B., Lu, K., and Bovin, N. (2009) Anti-carbohydrate antibodies of normal sera: findings, surprises and challenges, Mol. Immunol. 46, 3037-3049.
10. White-Scharf, M. E., and Rosenberg, L. T. (1978) Evidence that L-rhamnose is the antigenic determinant of hyporesponsiveness of BALB/c mice to Klebsiella pneumoniae type 47, Infect. Immun. 22, 18-21.
11. Prakobphol, A., and Linzer, R. (1980) Purification and immunological characterization of a rhamnose-glucose antigen from Streptococcus mutans 6517-T2 (serotype g), Infect. Immun. 30, 140-146.
12. Prakobphol, A., Linzer, R., and Genco, R. J. (1980) Purification and characterization of a rhamnosecontaining cell wall antigen of Streptococcus mutans B13 (serotype d), Infect. Immun. 27, 150-157.

13. Hanna, H. R., and Bundle, D. R. (1993) Antibody-oligosaccharide interactions: the synthesis of 2- deoxy-alpha -L-rhamnose containing oligosaccharide haptens related to Shigella flexneri variant Y antigen, Can. J. Chem. 71, 125-134.
14. Zhang, J., Zhu, Y., and Kong, F. (2001) Synthesis of an L-rhamnose tetrasaccharide, the common and major structure of the repeating unit of the O-antigenic polysaccharide of a strain of Klebsiella pneumoniae and Pseudomonas holci, Carbohydr. Res. 336, 229-235.
15. Seebeck, E., and Schindler, O. (1946) Isolation of an emodin bioside from the bark of Rhamnus frangula, Helv. Chim. Acta 29, 317-324.
16. Davis, B. G. (2002) Synthesis of glycoproteins, Chem. Rev. 102, 579-602.
17. Rostovtsev, V. V., Green, L. G., Fokin, V. V., and Sharpless, K. B. (2002) A stepwise Huisgen cycloaddition process: copper(I)-catalyzed regioselective “ligation” of azides and terminal alkynes, Angew. Chem., Int. Ed. 41, 2596-2599.
19. Bannwarth, W., and Knorr, R. (1991) Formation of carboxamides with N,N,N′,N′-tetramethyl-(succinimido)-uronium tetrafluoroborate in aqueous organic solvent systems, Tetrahedron Lett. 32, 1157-1160.
20. Andersson, M., Oscarson, S., and Ohberg, L. (1993) Synthesis of oligosaccharides with oligoethylene glycol spacers and their conversion into glycoconjugates using N,N,N′,N′-tetramethyl-(succinimido)-uronium tetrafluoroborate as coupling reagent, Glycoconj. J. 10, 461-465.
21. Lown, J. W., Rao Koganty, R., and Joshua, A. V. (1982) Synthesis of specifically deuterium labeled sulfur and oxygen ether side-chain-extended antileukemic (2-chloroethyl)nitrosoureas and study of their products and pathways of decomposition under physiological conditions, J. Org. Chem. 47, 2027-2033.
22. Hassane, F. S., Frisch, B., and Schuber, F. (2006) Targeted Liposomes: Convenient Coupling of Ligands to Preformed Vesicles Using “Click Chemistry”, Bioconjugate Chem. 17, 849-854.
23. Serrano, P., Casas, J., Llebaria, A., Zucco, M., Emeric, G., and Delgado, A. (2007) Parallel Synthesis and Yeast Growth Inhibition Screening of Succinamic Acid Libraries, J. Comb. Chem. 9, 635-643.

In the claims:

1. A pharmaceutical composition comprising a therapeutically effective amount of a T-cell antigen in covalent association with an L-Rhamnose or Forssman epitope.

2. The pharmaceutical composition of claim 1, wherein the composition comprises attenuated vaccine tumor cells, on which L-Rhamnose monosaccharide and/or Forssman disaccharide is present.

3. The pharmaceutical composition of claim 2, wherein said vaccine tumor cells are attenuated by gamma irradiation.

4. The pharmaceutical composition of claim 2, wherein said vaccine tumor cells are allogeneic, syngeneic, or autologous.

5. The pharmaceutical composition of claim 1, comprising one or more synthetic peptide antigens and a covalently associated L-Rhamnose monosaccharide or Forssman disaccharide via a chemical linker.

6. The pharmaceutical composition of claim 5, wherein the synthetic peptide antigen is a viral protein or peptide or a tumor antigen.

7. The pharmaceutical composition of claim 6, wherein the synthetic peptide antigen is a tumor antigen.

8. The pharmaceutical composition of claim 6, wherein the synthetic peptide antigen(s) include at least one antigen selected from an antigen of a human immunodeficiency virus, an influenza virus, a hepatitis B virus, a hepatitis C virus, a herpes simplex virus, or a human papilloma virus.

9. The pharmaceutical composition of claim 8, wherein at least one viral peptide antigen is a protein or peptide of a viral core, matrix, envelope, nucleoprotein, DNA or RNA ploymerase, integrase, or viral regulatory protein.

10. The pharmaceutical composition of claim 6, wherein the synthetic peptide antigen is a tumor antigen.

11. The composition of claim 5, wherein said chemical linker is an NHS-activated linker.

12. The composition of claim 5, wherein the chemical linker comprises one or more groups selected from alkyl, ether, polyether, amide and polyamide.

13. The composition of claim 1, further comprising a carrier or adjuvant.

14. The composition of claim 1, comprising an L-Rhamnose monosaccharide.

15. The composition of claim 1, comprising a Forssman disaccharide.

16. A method for inducing an immune mediated destruction of tumor cells, virus-infected cells, or virus in an animal comprising: administering to an animal in need thereof a composition of claim 1.
